(19) United States
(12) Patent Application Publication
(10) Pub. No.: US 2016/0115189 A1
(43) Pub. Date: Apr. 28, 2016

(54) PARASITICIDAL COMPOUNDS, METHODS, AND FORMULATIONS

(71) Applicant: ELI LILLY AND COMPANY, Indianapolis, IN (US)

(72) Inventors: Shu-Hui Chen, Indianapolis, IN (US); Jean Marie Defauw, Indianapolis, IN (US); Scott Dale Holmstrom, Indianapolis, IN (US); Shon Roland Pulley, Indianapolis, IN (US); William Hunter White, Indianapolis, IN (US); Joseph Raymond Winkle, Indianapolis, IN (US); Wentao Wu, Indianapolis, IN (US); Yang Zhang, Indianapolis, IN (US)

(73) Assignee: ELI LILLY AND COMPANY, Indianapolis, IN (US)

(21) Appl. No.: 14/890,743

(22) PCT Filed: Jun. 5, 2014
(86) PCT No.: PCT/US2014/041106

§ 371 Date: Nov. 12, 2015

(30) Foreign Application Priority Data
Jun. 6, 2013 (CN) PCT/CN2013/076846

(51) Int. Cl. C07H 17/08; A61K 31/706; A61K 31/7064; A61K 31/7048; A61K 9/00

(52) U.S. Cl. CPC C07H 17/08; A61K 31/7048; A61K 9/0014; A61K 31/7064; A61K 31/706

(57) ABSTRACT

Provided are compounds of formula I useful for controlling parasites both in animals and agriculture. Further provided are methods for controlling parasite infestations of an animal by administering an effective amount of a compound as described above, or a pharmaceutically acceptable salt thereof, to an animal, as well as formulations for controlling parasite infestations using the compounds described above or an acceptable salt thereof, and an acceptable carrier.

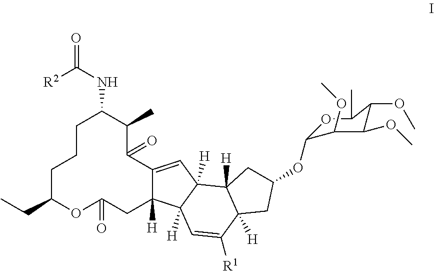

Ectoparasites such as fleas, lice, flies, mosquitoes, ticks and mites, as well as endoparasites such as gastrointestinal tract nematodes, flukes, and filarids, are problematic for man and animal alike. Such parasites seriously impact productivity in the domesticated animal industry by reducing weight gain, causing poor quality hide, wool, and meat, and in some cases resulting in death. Ecto- and endoparasites are also responsible, in part, for the spread of disease and discomfort in food and companion animals. Ectoparasites in particular are known to harbor and transmit a variety of microbial pathogens, including bacteria, viruses and protozoan parasites, many of which are pathogenic to humans, other warm-blooded mammals and birds. Diseases in which ectoparasites have been implicated include, but are not limited to, malaria, lymphatic- and blood-born filariasis, trachoma, trypanosomiasis, Leishmaniasis, Rocky Mountain Spotted Fever, Lyme Disease, babesiosis, and food-borne illnesses due to Salmonella, E. coli and Campylobacter, for example.

The medical importance of parasiticide infestations has prompted the development of reagents capable of controlling such. Commonly encountered methods to control parasiticidal infestation, for example, have generally focused on use of insecticides, which are often unsuccessful or unsatisfactory for one or more of the following reasons: (1) failure of owner or applicator compliance (frequent administration is required); (2) behavioral or physiological intolerance of the animal to the pesticide product or means of administration; (3) the emergence of ectoparasites resistant to the reagent; and (4) negative impact on the environment and/or toxicity.

Specifically, ticks parasitize wild as well as domesticated animals and humans, and are known or suspected to be responsible for the transmission of pathogens including bacteria, viruses and protozoan parasites. Currently, ticks are considered to be second in the world to mosquitoes as vectors of human diseases, but they are considered to be the most important vector of pathogens in North America. Effective elimination of tick infestations is difficult and often impractical, due to the need for concomitant treatment of the immediate host as well as the environmental reservoir. Presently, tick control is effected by integrated pest management in which different control methods are adapted to one area or against one tick species with due consideration to their environmental effects.

While the use of insecticides and pesticides have been beneficial, alternative or improved compounds, formulations, and methods are needed. Desirable compounds, formulations, and methods would not only provide alternative therapies, but would also overcome one or more of the limitations of current approaches. Such limitations include toxicity and safety of both the animal and the user/owner, limited efficacy (e.g., potency and duration), and resistance issues. Also impacting the beneficial use of insecticides and pesticides are administration obstacles, which include mode and recurrence of administration. For example, reducing the frequency of administration while maintaining efficacy is desirable, as excessive and repeated treatment of animals is often inconvenient and/or difficult.

The present invention encompasses parasiticidal compounds, methods, and formulations for use in and on animals and plants, and which provide alternative options for combating parasiticidal infestations, particularly ectoparasiticidal infestations. Further, certain aspects of the invention overcome at least some limitations in the use of current insecticides and pesticides, particularly in providing effective long term, safe control of parasites.

Provided are compounds, and salts thereof, of formula I:

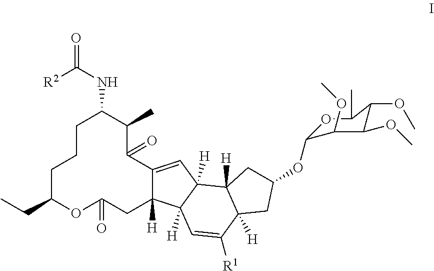

wherein R¹ is hydrogen or methyl; and R² is

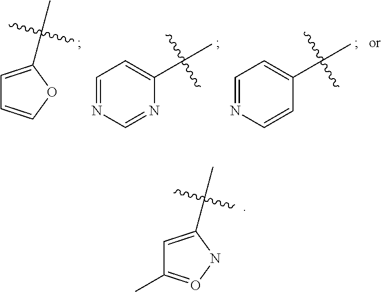

The invention provides a formulation, including a pharmaceutical formulation, comprising a compound of formula I, or salt thereof, and one or more acceptable carriers. The formulation may further comprise at least one additional active ingredient. A pharmaceutical formulation of the invention may be a human pharmaceutical formulation or a veterinary pharmaceutical formulation.

The invention provides a method of controlling ecto- and endoparasite infestations of an animal in need thereof comprising administering an effective amount of a compound formula I, or salt thereof, to the animal. The method may further provide administering at least one other active ingredient to said animal.

The present invention provides a method for preventing and treating diseases transmitted through parasites comprising administering at least one compound of the invention, or a salt thereof, to an animal in need thereof.

The invention provides a method for controlling parasites, characterized in that a compound of formula I, or a salt thereof, is allowed to act on the pests and/or their habitat. The invention provides the use of compounds of formula I, or salts thereof, for controlling pests.

The invention provides a compound of formula I, or a salt thereof, for use in therapy. The invention further provides a compound or salt of formula I for use in controlling ecto- and endoparasite infestations. The invention also provides use of a compound of formula I, or a salt thereof, for the manufacture of a formulation or medicament for controlling ecto- and endoparasite infestations.

The host animal may be a mammal or non-mammal, such as a bird (turkeys, chickens) or fish. Where the host animal is a mammal, it may be a human or non-human mammal. Non-human mammals include domestic animals, such as livestock animals and companion animals. Livestock animals include cattle, camellids, pigs, sheep, goats, and horses. Companion animals include dogs, rabbits, cats, and other pets owned and maintained in close association with humans as part of the human-animal bond.

Parasites, sometimes also referred to as pests, include both ectoparasites and endoparasites. Ectoparasites include insect and acarine pests which commonly infest or infect animals, and include the egg, larval, pupal, nymphal, and adult stages thereof. Such pests include fleas, lice, mosquitoes, mites, ticks, beetles, and blood-sucking, biting, or nuisance fly species. Endoparasites include nematode pests which commonly infect animals, and include the egg, larval, and adult stages thereof. Such pests include helminths (hookworms, tapeworms, heartworms), and are commercially important because they cause serious diseases in animals, e.g. in sheep, pigs, goats, cattle, horses, donkeys, camels, dogs, cats, rabbits, guinea-pigs, hamsters, chicken, turkeys, guinea fowls and other farmed birds, as well as exotic birds. Typical nematodes are Haemonchus, Trichostrcngyius, Qstertagia, Nematotiirus, Cooperia, Ascaris, Bunostonum, Gesophagostonum, Charbertia, Trichuris, Strongyius, Trïchonema, Dictyocaulus, Capsliarsa, Heterakis, Toxocara, Ascaridia, Oxyuris, Ancyiostoma, Uncinaria, Toxascaris and Parascaris. The trematodes include, in particular, the family of Fasciolideae, especially Fasciola hepatica.

Controlling refers to either ameliorating or eliminating a current infestation, or preventing an infestation, in or on an animal host or a plant.

Effective amount refers to the amount of a compound of formula I, or a salt thereof, sufficient to control an ecto- or endoparasite infestation, and includes causing a measurable reduction in the ecto- or endoparasite infestation population, and as such will depend upon several factors. For use on or in animals, ranges for a compound of formula I, or a salt thereof, in the methods include from 0.01 to 1000 mg/kg and more desirably, 0.1 to 100 mg/kg of the animal's body weight. The frequency of the administration will also be dependent upon several factors, and can be a single dose administered once a day, once a week, once a month, once every two, three, four, or six months, or for any duration as determined by a medical doctor, veterinarian, or other qualified medical or veterinary professional. Additional active ingredients may be administered with a compound of formula I.

Pharmaceutically acceptable as used in this application, for example with reference to salts and formulation components such as carriers, includes “dermatologically acceptable” and “veterinarily acceptable”, and thus includes both human and animal applications independently.

Salts of the compounds of the invention, including pharmaceutically acceptable salts, and common methodology for preparing them, are known in the art. See, e.g., P. Stahl, et al., HANDBOOK OF PHARMACEUTICAL SALTS: PROPERTIES, SELECTION AND USE, (VCHA/Wiley-VCH, 2002); S. M. Berge, et al., “Pharmaceutical Salts,” Journal of Pharmaceutical Sciences, Vol. 66, No. 1, January 1977.

The compounds of the invention and their salts may be formulated as pharmaceutical compositions for administration. Such pharmaceutical compositions and processes for making the same are known in the art for animals, including both humans and non-human mammals. See, e.g., REMINGTON: THE SCIENCE AND PRACTICE OF PHARMACY, (A. Gennaro, et al., eds., 19^{th} ed., Mack Publishing Co., 1995). Formulations can be administered through various means, including oral administration, parenteral administration such as injection (intramuscular, subcutaneous, intravenous, intraperitoneal) or the like; topical application with or without transdermal penetration such as dipping, spray, bathing, washing, pouring-on and spotting-on, and dusting, or the like. Additional active ingredients may be included in the formulation containing a compound of the invention or a salt thereof.

Carrier is used herein to describe any ingredient other than the active component(s) in a formulation. The choice of carrier will to a large extent depend on factors such as the particular mode of administration or application, the effect of the carrier on solubility and stability, and the nature of the dosage form.

Diseases transmitted through parasites, particularly ectoparasites such as ticks, are, for example bacterial, viral, rickettsial and protozoal vector-borne diseases. Examples of viral diseases transmitted through arboviruses, i.e. arthropod borne viruses, are Crimean-Congo Hemorhagic Fever (CCHF), Febrile illness, Papataci fever, Encephalitis, Meningitis, which are caused by Bunyaviridae such as Bunyavirus, Nairovirus or Phlebovirus; Bluetongue, meningoencephalits, Febrile illness, hemorhagic fever, which are caused by Reoviridae, such as Orbivirus, Colitivirus; Febrile illness, rash, encephalitis, polyarthritis, lymphadenitis, which are caused by Togaviridae, such as Sindbisvirus, Chikungunya Virus; tick-borne meningoencephalitis, Dengue hemorhagic fever, encephalitis, Febrile illness, Yellow fever, which are caused by Flaviviridae, such as Flavivirus (including diverse sub-groups). Examples of bacterial diseases transmitted through parasites are Rickettsiosis, such as Rocky Mountain spotted fever, tick typhus caused by infection through Rickettsia ssp; Tularemia caused by infection through Francisella tularensis; Borreliosis or Spirochaetosis, such as Lyme disease, or relapsing fever, caused, by infection through Borrelia ssp.; Ehrllichiosis caused by infection through Ehrlichia ssp.; Plague, caused by infection through Yersinia ssp. Examples of protozoal or rickettsial borne diseases are Babesiosis, such as Texas fever, red water disease, Q-fever caused by infection through Babesia ssp.; Theileriosis, such as east coast fever, Mediterranean coast fever, caused by infection through Theileria ssp.; Nagana disease, Sleeping sickness caused by infection through Trypanosoma ssp., Anaplasmosis caused by infection through Anaplasma ssp.; Malaria caused by infection through Plasmodium ssp.; Leishmaniasis caused by infection through Leishmania ssp.

Given their activity, certain of the compounds of the invention are suitable as soil pesticides against pests in the soil, as well as pesticides for plants, such as cereals, cotton, rice, maize, soya, potatoes, vegetables, fruit, tobacco, hops, citrus, and avocados. Certain compounds according to the invention are suitable for protecting plants and plant organs, for increasing the harvest yields, and for improving the quality of the harvested material which are encountered in agriculture, in horticulture, in forests, in gardens, and leisure facilities, and in the protection of stored products and of materials. They may be employed as plant protection agents.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out by conventional and known means, including directly acting on, or by allowing the compounds to act on, the surroundings, habitat or storage space by the customary treatment methods, for example by immersion, spraying, evaporation, fogging, scattering, painting on, injection and, in the case of propagation material, in particular in the case of seeds, also by applying one or more coats.

The compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and microencapsulations in polymeric substances.

These formulations are produced in a known manner, for example by mixing the active compounds with extenders, that is liquid solvents and/or solid carriers, optionally with the use of surfactants, that is emulsifiers and/or dispersants and/or foam-formers. The formulations are prepared in suitable plants or elsewhere before or during the application.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are extenders, solvents, and carriers.

Additional active ingredients may be included in the methods and formulations of the invention. Such additional active ingredient may be one or more additional compounds of the invention, or active ingredients outside the scope of the invention. For example, an additional active compound may be included to complement a compound of the invention, such as in terms of conveying improved spectrum or duration of activity. Such additional active ingredients include, but are not limited to, endoparasiticides belonging to the macrocyclic lactone (e.g., ivermectin, milbemycin, milbemycin oxime), benzimidazole (e.g., flubendazole), imidathioazole (e.g., levamisole), spiroindole (e.g., derquantel), piperazine, tribendimidine, salicylanilide (e.g., niclosamide), tetrahydropyrimidine (e.g., pyrantel), benzamide (e.g., closantel), cyclooctadepsipeptide (e.g., emodepside) or aminoacetonitrile derivative (e.g., monepantel) class as well as antiprotozal agents such as pentamidine, pyramethamine, suramin, nitazoxanide, and melarsoprol. An additional active ingredient may also be an ectoparasicidal or endectoparasiticidal compound including, but not limited to, a macrocyclic lactone (e.g., ivermectin, milbemycin, milbemycin oxime), spinosyn (e.g., spinosad, spinetoram), pyrazole or phenylpyrazole (e.g., fipronil, tebufenpyrad), formamidine (e.g., amitraz), neonicotinoid (e.g., imidacloprid, thiamethoxam), cyclodiene organochlorine (e.g., dieldrin, DDT), nodulasporamide, pthalamide (e.g., tetramethrin), pyrethroid (e.g., permethrin), diamide (e.g., chlorantraniliprole), oxadiazine (e.g., indoxicarb), organophosphate (e.g., diazinon), dinitrophenol (e.g., DNOC), carbamate (e.g., carbaryl), semicarbazone (e.g., metaflumizone), isoxazoline (e.g., fluralaner), pyrimidinamine (e.g., pyrimidifen), pyyrole (e.g., chlorfenapyr), tetramic acid (e.g., spirotetramet), and thiazole (e.g., clothianidin), as well as various unclassified parasiticides such as acequinocyl, pyridalyl, and members of the aminobenzamide-class of ectoparasiticides, and insect growth regulators (e.g., juvenile hormone mimics, chitinase inhibitors) such as pyrirpoxifen and S-methoprene.

Following are examples for preparing the compounds of the invention. The examples, and information contained therein, are illustrative, and can be modified in ways known in the art to obtain the desired results.

Spinosad, which may serve as the starting material for preparing the compounds of the invention, is comprised mainly of two spinosyn factor: A and D. Generally, spinosad comprises around 65-95% spinosyn A and 5-35% of spinosyn D. Accordingly, when using spinosad, the resultant compounds may be a combination of compounds of Formula I, where R₁ is hydrogen and methyl.

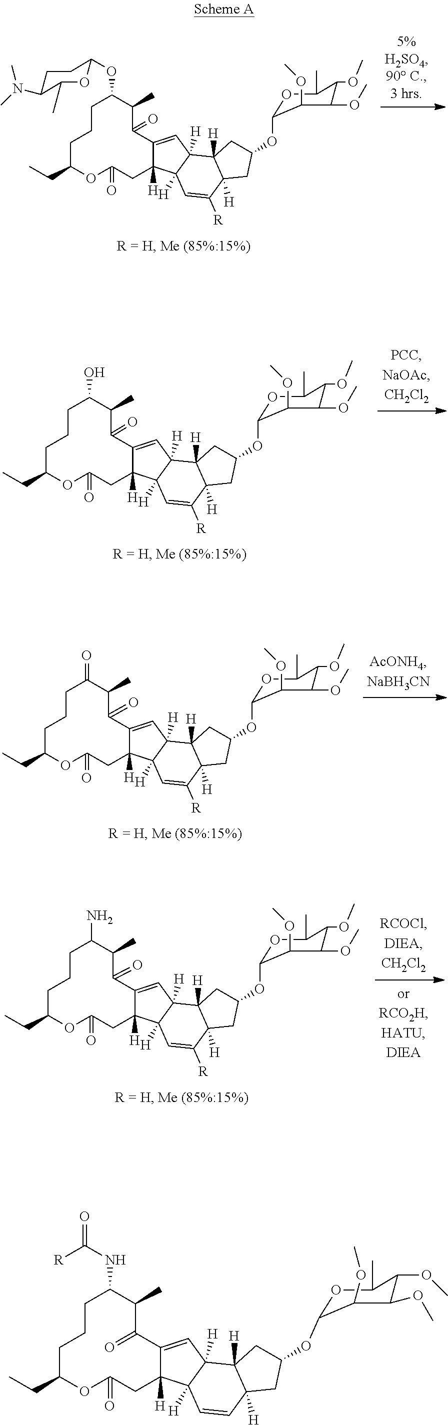

### Preparation 1

### Mixture of (2R,3aS,5aR,5bS,9S,13S,14R,16aS,16bR)-9-ethyl-13-hydroxy-14-methyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione and (2S,3aR,5aS,5bS,9S,13S,14R,16aS,16bS)-9-ethyl-13-hydroxy-4,14-dimethyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione (85:15)

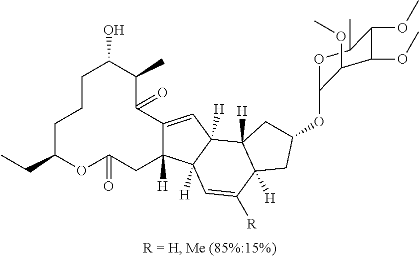

Stir a suspension of Spinosad A& D (85:15, 27.0 g, 36.89 mmol) in 5% H₂SO₄ (270 mL) at 90° C.-100° C. for 3 hours. After cooling down to room temperature, collect the precipitate by filtration. Wash the filter cake with water (3×20 mL) and dry in vacuo to afford a mixture of (2R,3aS,5aR,5bS,9S,13S,14R,16aS,16bR)-9-ethyl-13-hydroxy-14-methyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione and (2S,3aR,5aS,5bS,9S,13S,14R,16aS,16bS)-9-ethyl-13-hydroxy-4,14-dimethyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione (85:15) as a white solid (20.0 g, Yield 92.08%). MS (m/z): 613 (M+23) and 627 (M+23).

### Preparation 2

### Mixture of (2R,3aS,5aR,5bS,9S,14R,16aS,16bR)-9-ethyl-14-methyl-2-(((2R,3R,5S,6S)-3,4,5-trimethoxy-6-methyltetrahydro-2H-pyran-2-yl)oxy)-3,3a,5b,6,9,10,11,12,16a,16b-decahydro-1H-as-indaceno[3,2-d][1]oxacyclododecine-7,13,15(2H,5aH,14H)-trione and (2S,3aR,5aS,5bS,9S,14R,16aS,16bS)-9-ethyl-4,14-dimethyl-2-(((2R,3R,5S,6S)-3,4,5-trimethoxy-6-methyltetrahydro-2H-pyran-2-yl)oxy)-3,3a,5b,6,9,10,11,12,16a,16b-decahydro-1H-as-indaceno[3,2-d][1]oxacyclododecine-7,13,15(2H,5aH,14H)-trione (85:15)

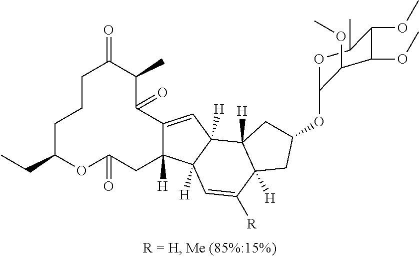

Stir a mixture of (2R,3aS,5aR,5bS,9S,13S,14R,16aS,16bR)-9-ethyl-13-hydroxy-14-methyl-2-(((2R,3R,5 S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione and (2S,3aR,5aS,5bS,9S,13S,14R,16aS,16bS)-9-ethyl-13-hydroxy-4,14-dimethyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione (85:15, 17.2 g, 28.5 mmol), PCC (18.1 g, 84.4 mmol), NaOAc (18.1 g, 221.1 mmol) in CH₂Cl₂ (850 mL) at room temperature overnight under N₂. Filter the mixture through a celite pad and wash the filtrate with brine (100 mL), dry over Na₂SO₄, filter and concentrate under reduced pressure to give a residue. Purify the residue by silica gel chromatography column (eluting with hexane:ethyl acetate=4:1) to afford a mixture of (2R,3aS,5aR,5bS,9S,14R,16aS,16bR)-9-ethyl-14-methyl-2-(((2R,3R,5S,6S)-3,4,5-trimethoxy-6-methyltetrahydro-2H-pyran-2-yl)oxy)-3,3a,5b,6,9,10,11,12,16a,16b-decahydro-1H-as-indaceno[3,2-d][1]oxacyclododecine-7,13,15(2H,5aH,14H)-trione and (2S,3aR,5aS,5bS,9S,14R,16aS,16bS)-9-ethyl-4,14-dimethyl-2-(((2R,3R,5S,6S)-3,4,5-trimethoxy-6-methyltetrahydro-2H-pyran-2-yl)oxy)-3,3a,5b,6,9,10,11,12,16a,16b-decahydro-1H-as-indaceno[3,2-d][1]oxacyclododecine-7,13,15(2H,5aH,14H)-trione (85:15) as a white solid (17.0 g, yield: 98.8%). MS (m/z): 611 (M+23) and 625 (M+23).

### Preparation 3

### Mixture of (2R,3aS,5aR,5bS,9S,14R,16aS,16bR)-13-amino-9-ethyl-14-methyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione and (2S,3aR,5aS,5bS,9S,14R,16aS,16bS)-13-amino-9-ethyl-4,14-dimethyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione (85:15)

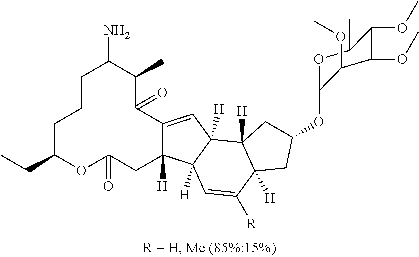

Adjust a co-solvent of 1,2-dichloroethane (70 mL) and MeOH (140 mL) to pH at 4 to 5 with the addition of AcOH. Then added a mixture of (2R,3aS,5aR,5bS,9S,14R,16aS,16bR)-9-ethyl-14-methyl-2-(((2R,3R,5S,6S)-3,4,5-trimethoxy-6-methyltetrahydro-2H-py ran-2-yl)oxy)-3,3a,5b,6,9,10,11,12,16a,16b-decahydro-1H-as-indaceno[3,2-d][1]oxacyclododecine-7,13,15(2H,5aH,14H)-trione and (2S,3aR,5aS,5bS,9S,14R,16aS,16bS)-9-ethyl-4,14-dimethyl-2-(((2R,3R,5S,6S)-3,4,5-trimethoxy-6-methyltetrahydro-2H-pyran-2-yl)oxy)-3,3a,5b,6,9,10,11,12,16a,16b-decahydro-1H-as-indaceno[3,2-d][1]oxacyclododecine-7,13,15(2H,5aH,14H)-trione (85:15, 4.34 g, 7.3 mmol), NH₄OAc (8.32 g, 108 mmol) and NaBH₃CN (1.13 g, 18 mmol) to the above co-solvent in turn. Stir the mixture at 50° C. for 16 hours under N₂. Then dilute the mixture with water (200 mL), wash with 10% aqueous NaHCO₃ solution, and extract with CH₂Cl₂ (100 mL×3). Combine the organic layers and wash with brine, dry over sodium sulfate, filter, and concentrate under reduced pressure to give a residue. Purify the residue by silica gel chromatography column (eluting with CH₂Cl₂:MeOH=10:1) to afford (2R,3aS,5aR,5bS,9S,14R,16aS,16bR)-13-amino-9-ethyl-14-methyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione and (2S,3aR,5aS,5bS,9S,14R,16aS,16bS)-13-amino-9-ethyl-4,14-dimethyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione (85:15) as white solid (2.93 g, yield: 67.6%, dr ratio is 2:1). MS (m/z): 590 (M+1) and 604 (M+1).

### EXAMPLE 1

### N-((2R,3aS,5aR,5bS,9S,13S,14R,16aS,16bR)-9-ethyl-14-methyl-7,15-dioxo-2-(((2R,3R,5S,6S)-3,4,5-trimethoxy-6-methyltetrahydro-2H-pyran-2-yl)oxy)-2,3,3a,5a,5 b,6,7,9,10,11,12,13,14,15,16a,16b-hexadecahydro-1H-as-indaceno[3,2-d][1]oxacyclododecin-13-yl)furan-2-carboxamide

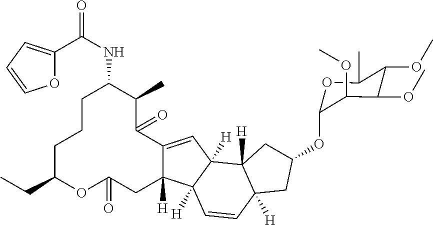 [SMILES: CC[C@H]1CCC[C@H](NC(=O)c2ccco2)[C@@H](C)C(=O)C2=C[C@@H]3[C@@H](C=C[C@@H]4C[C@@H](OC5OC(C)C(OC)C(OC)C5OC)C[C@@H]34)[C@@H]2CC(=O)O1]

Add dropwise a solution of furan-2-carbonyl chloride (7.02 g, 54.0 mmol) in CH₂Cl₂ (50 mL) to a mixture of (2R,3aS,5aR,5bS,9S,14R,16aS,16bR)-13-amino-9-ethyl-14-methyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione and (2S,3aR,5aS,5bS,9S,14R,16aS,16bS)-13-amino-9-ethyl-4,14-dimethyl-2-(((2R,3R,5S,6S)-3,4,5-trimethoxy-6-methyltetrahydro-2H-pyran-2-yl)ox y)-3,3a,5b,6,9,10,11,12,13,14,16a,16b-dodecahydro-1H-as-indaceno[3,2-d][1]oxacyclododecine-7,15(2H,5aH)-dione (85:15, 10.86 g, 18.4 mmol), DIEA (5.81 g, 45.0 mmol) in anhydrous CH₂Cl₂ (600 mL) at room temperature. Then stir the resultant mixture at 30° C. for 12 hours. Quench the reaction with water (200 mL) and neutralize the mixture with aqueous NaHCO₃ to pH at 7.0. Then extract the aqueous mixture with CH₂Cl₂ (200 mL×3). Combine the organic layers and extract with brine, dry over sodium sulfate, filter, and concentrate under reduced pressure to give a residue. Purify the residue by acidic preparative HPLC first, then followed by SFC separation to afford N-((2R,3aS,5aR,5bS,9S,13S,14R,16aS,16bR)-9-ethyl-14-methyl-7,15-dioxo-2-(((3R,4R,6S)-3,4,5-trimethoxy-6-methyltetrahydro-2H-pyran-2-yl)oxy)-2,3,3a,5a,5b,6,7,9,10,11,12,13,14,15,16a,16b-hexadecahydro-1H-as-indaceno[3,2-d][1]oxacyclododecin-13-yl)furan-2-carboxamide as a white powder (4.08 g, yield: 32.5%). MS (m/z): 684 (M+1). ¹H NMR (400 MHz, CDCl₃-d) δ 0.81-0.86 (m, 3H), 1.17 (d, J=6.8 Hz, 3H), 1.28 (d, J=6.3 Hz, 6H), 1.45-1.67 (m, 7H), 1.92 (dd, J=13.3 & 7.0 Hz, 1H), 2.15-2.27 (m, 2H), 2.47 (dd, J=14.4 & 2.6 Hz, 1H), 2.81 (dd, J=11.1 & 8.9 Hz, 1H), 3.04 (d, J=4.7 Hz, 1H), 3.09-3.23 (m, 2H), 3.34-3.40 (m, 1H), 3.50 (d, J=2.7 Hz, 9H), 3.56 (s, 4H), 3.73 (d, J=7.0 Hz, 1H), 4.19-4.36 (m, 2H), 4.71-4.80 (m, 1H), 4.85 (s, 1H), 5.78-5.83 (m, 1H), 5.86-5.92 (m, 1H), 6.39 (d, J=9.5 Hz, 1H), 6.53 (dd, J=3.1 & 1.6 Hz, 1H), 6.70 (br. s., 1H), 7.15 (d, J=3.3 Hz, 1H), 7.47 (s, 1H).

### EXAMPLE 2

### N-((2R,3aS,5aR,5bS,9S,14R,16aS,16bR)-9-ethyl-14-methyl-7,15-dioxo-2-(((2R,3R,5S,6S)-3,4,5-trimethoxy-6-methyltetrahydro-2H-pyran-2-yl)oxy)-2,3,3a,5a,5 b,6,7,9,10,11,12,13,14,15,16a,16b-hexadecahydro-1H-as-indaceno[3,2-d][1]oxacyclododecin-13-yl)pyrimidine-4-carboxamide

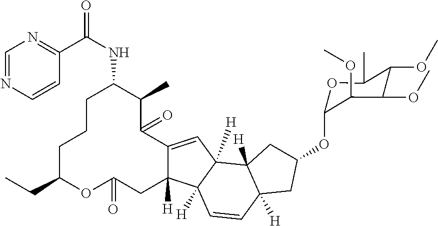 [SMILES: CC[C@H]1CCC[C@H](NC(=O)c2ccncn2)[C@@H](C)C(=O)C2=C[C@@H]3[C@@H](C=C[C@@H]4C[C@@H](OC5OC(C)C(OC)C(OC)C5OC)C[C@@H]34)[C@@H]2CC(=O)O1]

Add dropwise a mixture of (2R,3aS,5aR,5bS,9S,14R,16aS,16bR)-13-amino-9-ethyl-14-methyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione and (2S,3aR,5aS,5bS,9S,14R,16aS,16bS)-13-amino-9-ethyl-4,14-dimethyl-2-(((2R,3R,5S,6S)-3,4,5-trimethoxy-6-methyltetrahydro-2H-pyran-2-yl)oxy)-3,3a,5b,6,9,10,11,12,13,14,16a,16b-dodecahydro-1H-as-indaceno[3,2-d][1]oxacyclododecine-7,15(2H,5aH)-dione (85:15.5 g, 8.49 mmol) in CH₂Cl₂ (40 mL) to a mixture of pyrimidine-4-carboxylic acid (2.09 g, 17.13 mmol), HATU (6.45 g, 16.96 mmol) and DIPEA (2.75 g, 21.32 mmol) in DMF (40 mL) at ambient temperature. Then heat the mixture to 55° for 1 hour. After cooling the mixture to room temperature, filter the reaction mixture, and concentrate the filtrate under reduced pressure to give a brown solid. Purify the residue by acidic preparative HPLC first, then followed by SFC separation to afford N-((2R,3aS,5aR,5bS,9S,14R,16aS,16bR)-9-ethyl-14-methyl-7,15-dioxo-2-(((2R,3R,5S,6S)-3,4,5-trimethoxy-6-methyltetrahydro-2H-pyran-2-yl)oxy)-2,3,3a,5a,5b,6,7,9,10,11,12,13,14,15,16a,16b-hexadecahydro-1H-as-indaceno[3,2-d][1]oxacyclododecin-13-yl)pyrimidine-4-carboxamide (1.92 g, yield: 32.6%) as brown solid. MS (m/z): 718 (M+23). ¹H NMR (400 MHz, CDCl₃) δ 0.83 (t, J=7.5 Hz, 3H) 0.90-1.02 (m, 1H) 1.17 (d, J=6.8 Hz, 3H) 1.26-1.36 (m, 6H) 1.48-1.57 (m, 4H) 1.94 (dd, J=13.4 & 7.0 Hz, 1H) 2.19-2.31 (m, 2H) 2.47 (dd, J=13.7 & 3.1 Hz, 1H) 2.89 (dd, J=11.5 & 8.8 Hz, 1H) 3.08 (br. s., 1H) 3.13 (t, J=9.4 Hz, 1H) 3.21 (dd, J=13.7 & 5.1 Hz, 1H) 3.39 (dd, J=9.9 & 6.8 Hz, 1H) 3.45-3.61 (m, 16H) 4.30-4.35 (m, 2H) 4.73 (d, J=7.1 Hz, 1H) 4.87 (d, J=1.1 Hz, 1H) 5.81-5.85 (m, 1H) 5.89-5.92 (m, 1H) 6.80 (br. s., 1H) 7.93 (d, J=9.9 Hz, 1H) 8.20 (dd, J=5.1 & 1.3 Hz, 1H) 9.02 (d, J=5.1 Hz, 1H) 9.28 (d, J=1.1 Hz, 1H).

The following compounds may be prepared essentially by the methods of Example 1 or Example 2.

| Ex. | | | |
|---|---|---|---|
| No. | Chemical name | Structure | Physical data |
| 3 | N-((2R,3aS,5aR,5bS, 9S,13S,14R,16aS, 16bR)-9-ethyl-14- methyl-7,15-dioxo- 2-(((2R,3R,5S,6S)- 3,4,5-trimethoxy- 6-methyltetrahydro- 2H-pyran-2-yl)oxy)- 2,3,3a,5a,5b,6,7, 9,10,11,12,13,14, 15,16a,16b-hexa decahydro-1H-as- indaceno[3,2-d][1] oxacyclododecin- 13-yl)isonicolina- mide | 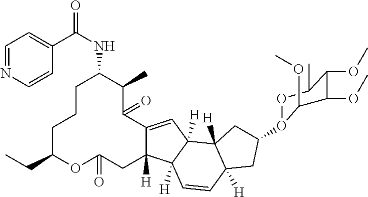 | MS (m/z): 695 (M + 1). ¹H NMR (CDCl₃, 400 MHz) δ 0.87 (t, J = 7.4 Hz, 4 H), 1.07-1.19 (m, 5 H), 1.21- 1.36 (m, 5 H), 1.45-1.53 (m, 3 H), 1.89 (dd, J = 13.3 & 7.0 Hz, 1 H), 2.12 (dd, J = 12.9 & 6.4 Hz, 2 H), 2.48 (br. s., 1 H), 2.59 (d, J = 17.1 Hz, 1 H), 3.02 (d, J = 5.3 Hz, 1H), 3.06- 3.12 (m, 1 H), 3.16 (dd, J = 16.9 & 4.6 Hz, 1 H), 3.40-3.57 (m, 16 H), 4.14 (t, J = 10.0 Hz, 1 H), 4.26 (q, J = 6.6 Hz, 1 H), 4.82 |
| | | | (s, 1 H), 4.90 (d, J = 5.5 |
| | | | Hz, 1 H), 5.78 (d, J = 9.8 |
| | | | Hz, 1 H), 5.89 (d, J = 9.8 |
| | | | Hz, 1 H), 6.40 (br. s., 1 H), |
| | | | 7.04 (d, J = 9.0 Hz, 1 H), |
| | | | 7.84 (d, J = 5.3 Hz, 2 H), |
| | | | 8.81 (d, J = 4.8 Hz, 2 H). |
| 4 | N-((2R,3aS,5aR,5bS, 9S,13S,14R,16aS, 16bR)-9-ethyl-14- methyl-7,15-dioxo- 2-(((2R,3R,5S,6S)- 3,4,5-trimethoxy- 6-methyltetrahydro- 2H-pyran-2-yl)oxy)- 2,3,3a,5a,5b,6,7,9, 10,11,12,13,14,15, 16a,16b-hexa decahydro-1H-as- indaceno[3,2-d][1] oxacyclododecin- 13-yl)-5-methyl- isoxazole-3- | 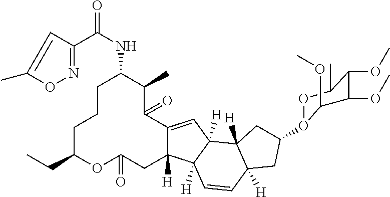 | MS (m/z): 699 (M + 1). ¹H NMR (CDCl₃, 400 MHz) δ 0.83 (t, J = 7.4 Hz, 3 H), 0.93 (dd, J = 11.3 & 6.8 Hz, 1 H), 1.17 (d, J = 6.8 Hz, 3H), 1.23-1.42 (m, 6 H), 1.43-1.77 (m, 7 H), 1.94 (dd, J = 13.4 & 6.9 Hz, 1 H), 2.02-2.35 (m, 3 H), 2.36- 2.52 (m, 5 H), 2.83-2.94 (m, 1 H), 3.06 (br. s., 1 H), 3.10-3.16 (m, 1 H), 3.19 (dd, J = 13.8 & 4.8 Hz, 1 H), 3.29-3.37 (m, 1 H), 3.45-3.62 (m, 12 H), 4.32 (d, J = 6.02 Hz, 2 H), 4.72 |
| | carboxamide | | (br. s., 1 H), 4.86 (s, 1 H), |
| | | | 5.77-5.85 (m, 1 H), 5.87- |
| | | | 5.93 (m, 1 H), 6.47 (s, 1 H), |
| | | | 6.68-6.81 (m, 2 H). |

### Assay 1: In Vitro Larval Immersion Microassay (LIM)

The larval immersion microassay may be conducted as described in White, et al., J. Med. Entomol. 41: 1034-1042 (2004). Briefly, experimental test articles are formulated in dimethylsulfoxide (DMSO) to prepare a stock solution at a concentration of 10 mM. Using 96-well microtiter plates, an aliquot of the 10 mM sample is subsequently diluted in a water-based solution containing 1% ethanol and 0.2% Triton X-100, to obtain the desired concentration (typically 0.3 mM or lower) of experimental test article in a volume of 0.1 ml (minimum of n=3 replicates per compound or concentration). Approximately 30-50 Lone star tick larvae (Amblyomma americanum) are submerged into each well containing experimental test articles. After a 30 minute immersion period, larvae are removed with a wide-bore pipette tip in 0.05 ml of fluid, dispensed into a commercial paper tissue biopsy bag which is sealed at the top with a plastic dialysis clip, inverted and allowed to air dry for 60 minutes. Bags containing larvae are then incubated at approximately 27 degrees Celsius and >90% relative humidity. After 24 hours, bags are opened, live and dead larvae are counted and percent larval efficacy is calculated as follows: % Efficacy=(# dead larvae)/(# total larvae)×100.

Example 1-4 compounds exhibit activity in this assay, and at the level of >50% efficacy when tested at a concentration of no greater than 0.3 mM.

### Assay 2: In Vivo Rodent Acaricide Test (RAT)

Evaluation of experimental test articles may be conducted using a modified version of the assay as described in Gutierrez et al., J. Med. Entomol. 43(3): 526-532 (2006). The assay is modified by using a different tick species: Dermacentor variabilis (the reference describes Amblyomma americanum ticks). Tick containment units (comprised of a baby nipple, ventilated screw cap top and reinforcing rubber washer) are attached to the shaved dorsum of adult Sprague-Dawley rats. After attachment of containment units, test materials are formulated in acetone to one or more desired concentrations (e.g., 1.0%, equivalent to 10 mg/ml) and applied directly to the surface of the skin enclosed by the containment unit, in a volume of 0.05 ml. Negative control rats are treated with 0.05 ml of acetone alone. After 4-6 hours, approximately ten (10) unfed, nymphal-stage American dog ticks (Dermacentor variabilis) are placed inside of each containment unit, which is sealed with a ventilated screw cap in order to prevent escape of tick nymphs. A minimum of three (3) and a maximum of five (5) rats are utilized per treatment group. Forty-eight (48) hours after infestation, containment units are removed and live and dead ticks are counted. Live tick counts are transformed using the natural logarithm transformation plus one (Ln count+1); addition of one to each count serve to adjust for counts that are zero. Geometric mean (GM) group tick counts are obtained via back-transformation of group mean transformed counts and subtracting one. The acetone-only control group is used for comparison to the groups receiving experimental test materials for the calculation of percent efficacy (% reduction in live tick counts).

The efficacy of treatments is calculated by comparing the geometric mean (GM) number of live ticks observed on treated rats with the GM number of live ticks counted on the negative control rats, using the following formula:

$$\% Efficacy = \frac{G M \# live ticks control - G M \# live ticks treated}{GM \# live ticks control} \times 100$$

Example 1-4 compounds exhibit activity (% efficacy) ≧50% at a topical application concentration of ≦1% active ingredient (10 mg/ml).

### Assay 3: In Vivo Adult Brown Tick and Cat Flea Infestations

The therapeutic (knockdown) and residual efficacy of the Example 1 compound, administered topically at a point dose of 30 mg/kg bodyweight, is evaluated against adult brown dog tick (Rhipicephalus sanguineus) and cat flea (Ctenocephalides felis) infestations on dogs. Based on pre-treatment tick retention rates, eight (8) beagle dogs are allocated to one of two treatment groups (n=4 per group): Untreated control group, and Example 1 compound-treated group. Dogs are infested with approximately 50 unfed, adult ticks one day prior to treatment (approximately 50% male and 50% female ticks). On Day 0, dogs are treated with Example 1 compound (50 mg/ml, dissolved in a vehicle solution of propylene carbonate, benzyl alcohol, and isopropyl myristate), applied via topical spot-on to achieve a point dose of 30 mg/kg. On Day 3 (approximately 72 hours after treatment), ticks are counted and removed from all dogs. To evaluate residual activity, dogs are re-infested with ticks on an approximate weekly basis for at least two weeks and up to 4 weeks, with tick counts (and removal) conducted 48 hours after each infestation. Dogs are infested with approximately 100 unfed, adult fleas at weeks 2 and 3 for both groups, concurrently with tick infestation.

The total number of live parasites present on each dog is determined for each interval, and this number is transformed using the natural logarithm transformation plus one (Ln count+1); addition of one to each count served to adjust for counts that are zero. Geometric mean (GM) group tick and flea counts are obtained via back-transformation of group mean transformed counts and subtracting one. The negative control group is used for comparison to treated groups for the calculation of percent efficacy (% reduction in live parasite counts). GM percent efficacy of treatments is calculated using the following formula:

$$\% Efficacy = \frac{\begin{matrix}G M No . Live Parasites Control - \\ G M No . Live Parasites Treated\end{matrix}}{G M No . Live Paraites Control} \times 100$$

As shown in Table 1, the residual efficacy of the Example 1 compound against ticks and fleas remains >90% through week 4. Treatments are well tolerated by all dogs.

TABLE 1
Geometric Mean Group Live Parasite Counts (% Efficacy)

| | Day 3 | Week 1 | Week 2 | Week 2 | Week 3 | Week 3 | Week 4 |
|---|---|---|---|---|---|---|---|
| Parasite: | Tick | Tick | Tick | Flea | Tick | Flea | Tick |
| Negative | 31.40 | 33.98 | 14.48 | 86.50 | 24.16 | 86.17 | 27.68 |
| Control | (—) | (—) | (—) | (—) | (—) | (—) | (—) |
| Example 1 | 3.95 | 0.19 | 0.00 | 7.15 | 0.00 | 7.21 | 0.00 |
| 30 mg/kg | (87.42) | (99.44) | (100) | (91.73) | (100) | (91.64) | (100) |

### Assay 4: In Vivo Adult Brown Tick and American Dog Tick Infestations

The therapeutic (knockdown) and residual efficacy of the Example 1 compound, administered via topical spot-on at descending point doses of 30, 20 and 10 mg/kg, is evaluated against adult tick (Dermacentor variabilis (American dog tick) and Rhipicephalus sanguineus (Brown dog tick)) infestations on dogs. Twenty-four (24) beagle dogs are allocated to either an untreated, negative control group or one of three treated groups (n=6 dogs per group). Dogs are infested with approximately 50 unfed, adult R. sanguineus ticks on Days −1, 5, 12, 19 and 28; dogs are concurrently infested with D. variabilis ticks on Day 12 and Day 28. On Day 0, dogs are treated with the Example 1 compound (50 mg/ml of technical active dissolved in a vehicle solution of propylene carbonate, benzyl alcohol, and isopropyl myristate), applied via topical spot-on in a volume so as to achieve point doses of 30, 20 and 10 mg/kg. Tick counts and classification are conducted on Days 3, 7, 14, 21 and 30. GM percent efficacy of treatments against both tick species is calculated using the formula in Assay 3.

Efficacy results against ticks are illustrated in Table 2. Therapeutic (knockdown) efficacy of treatments on Day 3 range from 73-85%. Residual efficacy against R. sanguineus was >95% through Day 21 for all treatments, and >95% through Day 30 for the 30 mg/kg dose. Residual efficacy against D. variabilis ticks is >95% for all doses at both Day 14 and Day 30 intervals. Treatment is well tolerated by all dogs.

TABLE 2
Geometric Mean Group Live Tick Counts (% Efficacy)*

| Tick | Day 3 | Day 7 | Day 14 | | Day 21 | Day30 | |
|---|---|---|---|---|---|---|---|
| Species: | Rs | Rs | Rs | Dv | Rs | Rs | Dv |
| Negative | 18.50 | 11.34 | 28.96 | 24.12 | 18.09 | 12.64 | 34.03 |
| Control | (—) | (—) | (—) | (—) | (—) | (—) | (—) |
| 30 mg/kg | 3.86 | 0.12 | 0.26 | 0.47 | 0.44 | 0.51 | 0.70 |
| | (79.13) | (98.92) | (99.10) | (98.06) | (97.55) | (95.94) | (97.95) |
| 20 mg/kg | 2.81 | 0.00 | 0.12 | 0.51 | 0.12 | 0.74 | 0.91 |
| | (84.80) | (100) | (99.58) | (97.87) | (99.32) | (94.12) | (97.34) |
| 10 mg/kg | 4.92 | 0.00 | 0.70 | 0.12 | 0.70 | 1.18 | 1.60 |
| | (73.39) | (100) | (97.59) | (99.49) | (96.14) | (90.64) | (95.30) |

*Rs = R. sanguineus, and Dv = D. variabilis

### Assay 5: In Vivo Adult Brown Tick Infestations

The therapeutic (knockdown) and residual efficacy of the Example 1-4 compounds, when administered via topical spot-on at 10 mg/kg, is evaluated against adult brown tick (Rhipicephalus sanguine us) infestations on dogs. Twenty (20) beagle dogs are allocated to either an untreated, negative control group or one of four treated groups (n=4 dogs per group). Dogs are infested with approximately 50 unfed, adult R. sanguineus ticks on Days −1, 5, 12, 19 and 28. On Day 0, dogs are treated with Example 1-4 compound formulations (each, 50 mg/ml of technical active dissolved in a vehicle solution of propylene carbonate, benzyl alcohol, and isopropyl myristate), applied via topical spot-on in a volume so as to achieve a point dose of 10 mg/kg. Tick counts and classification are conducted on Days 2, 7, 14, 21 and 30. GM percent efficacy of treatments against ticks is calculated using the formula in Assay 3.

Efficacy results are illustrated in Table 3. Therapeutic (knockdown) efficacy is similar for all 4 treatments on Day 2 (54-68%). Residual efficacy against R. sanguineus is >95% through Day 7 for Example compounds 1, 2, and 4, and remains >95% through Day 21 for the Example 1 compounds. All treatments are well tolerated by dogs.

TABLE 3

| | Day 2 | Day 7 | Day 14 | Day 21 | Day 30 |
|---|---|---|---|---|---|
| | Tick Species: | | | | |
| | R. sanguineus | R. sanguineus | R. sanguineus | R. sanguineus | R. sanguineus |
| Negative | 31.49 | 21.54 | 21.78 | 33.60 | 26.22 |
| Control | (—) | (—) | (—) | (—) | (—) |
| Ex. 3 | 11.80 | 3.23 | 5.12 | 5.90 | 15.47 |
| 10 mg/kg | (62.52) | (85.01) | (76.50) | (82.44) | (41.02) |
| Ex. 2 | 14.55 | 0.19 | 5.82 | 7.91 | 17.31 |
| 10 mg/kg | (53.80) | (99.12) | (73.29) | (76.46) | (33.99) |
| Ex. 1 | 10.03 | 0.00 | 0.57 | 1.59 | 8.40 |
| 10 mg/kg | (68.16) | (100) | (97.41) | (95.27) | (67.97) |
| Ex. 4 | 10.17 | 0.73 | 3.86 | 6.34 | 19.17 |
| 10 mg/kg | (67.71) | (96.60) | (82.25) | (81.13) | (26.88) |

### Assay 6: Efficacy Against Cattle Ticks (Rhipicephalus (Boophilus) Microplus)

Certain compounds of this invention may be useful for the control of ectoparasite infestations afflicting food producing animals, particularly ticks. Evaluation of activity against cattle tick or southern cattle tick (Rhipicephalus (Boophilus) microplus and/or annulatus) may be conducted using published vitro bioassay methods (larval packet test or larval immersion microassay; Miller et al., 2011, J. Med. Entomol. 48: 358-365). Activity of Compounds may also be evaluated against experimental or natural tick infestations on cattle using routine methods published in the literature (Holdsworth, P. A., et al., 2006, Vet. Parasitol. 136: 29-43).

We claim:

1. A compound, or a salt thereof, of Formula I 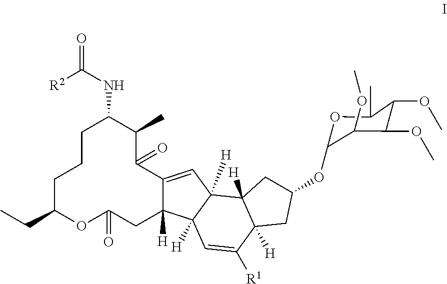
wherein R¹ is hydrogen or methyl; and R² is

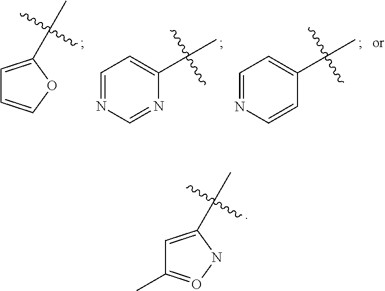

2. The compound of claim 1, wherein R¹ is hydrogen.

3. The compound of claim 1, wherein R¹ is methyl.

4. The compound of any of claims 1-3, wherein R² is

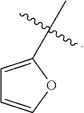 [SMILES: *c1ccco1]

5. The compound of any of claims 1-3, wherein R² is

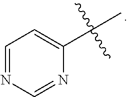 [SMILES: *c1ccncn1]

6. The compound of any of claims 1-3, wherein R² is

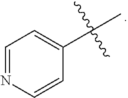 [SMILES: *c1ccncc1]

7. The compound of any of claims 1-3, wherein R² is

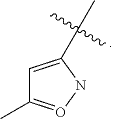

8. The compound of claim 1, wherein it is

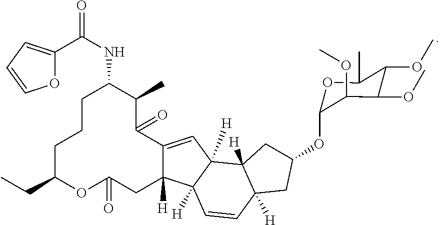 [SMILES: CC[C@H]1CCC[C@H](NC(=O)c2ccco2)[C@@H](C)C(=O)C2=C[C@@H]3[C@@H](C=C[C@@H]4C[C@@H](OC5OC(C)C(OC)C(OC)C5OC)C[C@@H]34)[C@@H]2CC(=O)O1]

9. The compound of claim 1, wherein it is

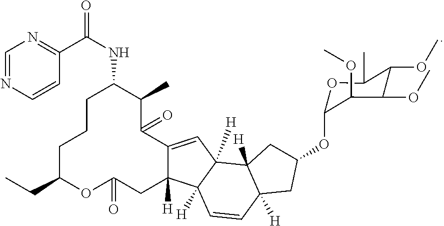 [SMILES: CC[C@H]1CCC[C@H](NC(=O)c2ccncn2)[C@@H](C)C(=O)C2=C[C@@H]3[C@@H](C=C[C@@H]4C[C@@H](OC5OC(C)C(OC)C(OC)C5OC)C[C@@H]34)[C@@H]2CC(=O)O1]

10. The compound of claim 1, wherein it is

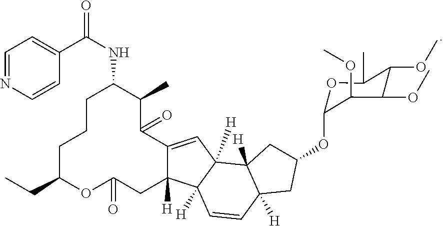 [SMILES: CC[C@H]1CCC[C@H](NC(=O)c2ccncc2)[C@@H](C)C(=O)C2=C[C@@H]3[C@@H](C=C[C@@H]4C[C@@H](OC5OC(C)C(OC)C(OC)C5OC)C[C@@H]34)[C@@H]2CC(=O)O1]

11. The compound of claim 1, wherein it is

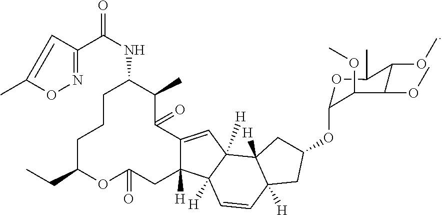

12. A composition comprising two compounds, or salts thereof, of Formula I 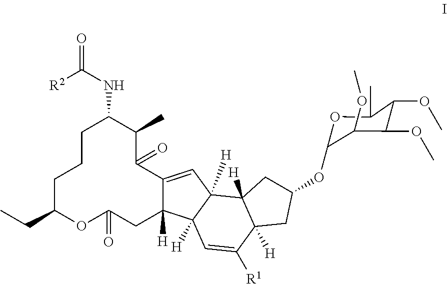
wherein R² is selected from 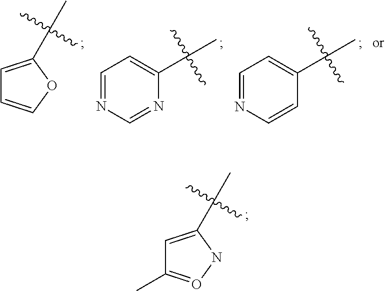
and
wherein the two compounds of Formula I include a compound where R¹ is hydrogen, and a compound where R¹ is methyl.

13. The composition of claim 12, wherein R² is 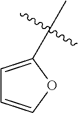 [SMILES: *c1ccco1]
in the two compounds of Formula I.

14. A formulation comprising a compound of any of claims 1-11, or salt thereof, or the composition of any of claims 12-13, and one or more acceptable carriers.

15. The formulation of claim 14, wherein it is a human pharmaceutical formulation.

16. The formulation of claim 14, wherein it is a veterinary pharmaceutical formulation.

17. The formulation of any of claims 14-16, wherein said formulation is for topical application.

18. The formulation of any of claims 14-17, wherein said formulation has at least one more additional active ingredient therein.

19. A method of controlling a parasite infestation in or on an animal in need thereof comprising administering an effective amount of a compound of any of claims 1-11, or salt thereof, or the composition of any of claims 12-13, to said animal.

20. The method of claim 19, wherein at least one other active ingredient is administered to said animal.

21. The method of claim 19 or claim 20, wherein said animal is a human.

22. The method of claim 19 or claim 20, wherein said animal is a companion animal.

23. The method of claim 22, wherein said companion animal is a dog or cat.

24. The method of claim 19 or 20, wherein said animal is a livestock animal.

25. The method according to any of claims 19-24, wherein said parasite is a tick.

26. The method according to any of claims 19-25, wherein said compound is administered topically.

27. A method for preventing or treating diseases transmitted through parasites comprising administering an effective amount of a compound of any of claims 1-11, or a salt thereof, or the composition of any of claims 12-13, to an animal in need thereof.

28. The method of claim 27, wherein at least one other active ingredient is administered to said animal.

29. The method of claim 27 or claim 28, wherein said animal is a human.

30. The method of claim 27 or claim 28, wherein said animal is a companion animal.

31. The method of claim 30, wherein said companion animal is a dog or cat.

32. The method of claim 27 or 28, wherein said animal is a livestock animal.

33. The method according to any of claims 27-32, wherein said parasite is a tick.

34. The method according to any of claims 27-33, wherein said compound is administered topically.

35. A method for controlling parasites, characterized in that a compound of any of claims 1-11, or a salt thereof, or the composition of any of claims 12-13, is allowed to act on the pests or their habitat, or both.

36. The method of claim 35, wherein the compound or the composition is placed on a plant or an animal.

37. Use of compounds, or salts thereof, of any of claims 1-11, or the composition of any of claims 12-13, for controlling parasites.

38. The use of claim 37, wherein at least one other active ingredient is used.

39. The use of claim 37 or 38, wherein said parasite is in or on an animal.

40. The use of claim 39, wherein said animal is a human.

41. The use of claim 39, wherein animal is a companion animal.

42. The use of claim 41, wherein said companion animal is a dog or cat.

43. The use of claim 39, wherein said animal is a livestock animal.

44. The use according to any of claims 37-43, wherein said parasite is a tick.

45. The use according to any of claims 39-44, wherein said compound or composition is administered topically.

46. A compound, or salt thereof, according to any of claims 1-11, or the composition of any of claims 12-13, for use in therapy.

47. A compound, or salt thereof, according to any of claims 1-11, or the composition of any of claims 12-13, for use in controlling a parasite infestation.

48. The compound, or salt thereof, or the composition of claim 47, wherein at least one other active ingredient is used.

49. The compound, or salt thereof, or the composition of claim 47 or 48, wherein said parasite is in or on an animal.

50. The compound, or salt thereof, or the composition of claim 49, wherein said animal is a human.

51. The compound, or salt thereof, or the composition of claim 49, wherein animal is a companion animal.

52. The compound, or salt thereof, or the composition of claim 51, wherein said companion animal is a dog or cat.

53. The compound, or salt thereof, or the composition of claim 49, wherein said animal is a livestock animal.

54. The compound, or salt thereof, or the composition according to any of claims 47-53, wherein said parasite is a tick.

55. The compound, or salt thereof, or the composition according to any of claims 47-54, wherein said compound is administered topically.
